(19) United States
(12) Patent Application Publication
(10) Pub. No.: US 2026/0245211 A1
(43) Pub. Date: Aug. 20, 2026

(54) INFORMATION PROCESSING SYSTEM, METHOD FOR CONTROLLING INFORMATION PROCESSING SYSTEM, AND COMPUTER-READABLE RECORDING MEDIUM

(71) Applicant: KYOCERA Corporation, Kyoto-shi, Kyoto (JP)

(72) Inventors: Kenichi WATANABE, Kyoto-shi, Kyoto (JP); Masayuki KYOMOTO, Kyoto-shi, Kyoto (JP); Kentaro KAMEI, Kyoto-shi, Kyoto (JP)

(21) Appl. No.: 19/159,451

(22) PCT Filed: Feb. 28, 2024
(86) PCT No.: PCT/JP2024/007350

§ 371 Date: Aug. 25, 2025

(30) Foreign Application Priority Data
Feb. 28, 2023 (JP) 2023-030325
Feb. 28, 2023 (JP) 2023-030326

(51) Int. Cl. G06T 7/00; G06V 10/776; G06V 10/82

(52) U.S. Cl. CPC G06T 7/0016; G06V 10/776; G06V 10/82; G06T 2207/20081; G06T 2207/20084; G06T 2207/30008; G06V 2201/033

(57) ABSTRACT

Bone state information on a future bone state of a subject is highly accurately estimated. An information processing system includes: an acquirer that acquires medical information including first medical information including a medical image showing a bone of a subject and second medical information including feature information of the subject at a time point different from a time point at which the medical image is captured; and an estimator that estimates bone state information on a future or past bone state of the subject by inputting the medical information into a learning model trained using training data including a plurality of pieces of information each including a medical image showing a bone of a predetermined person and feature information of the predetermined person at a time point different from a time point at which the medical image is captured.

### TECHNICAL FIELD

The present disclosure relates to an information processing system, a terminal device, a method for controlling the information processing system, and the like for estimating bone state information on a future bone condition of a subject.

### BACKGROUND OF INVENTION

Patent Document 1 discloses a technique for outputting information on a future symptom of a joint of a subject using a prediction model.

### CITATION LIST

### Patent Literature

Patent Document 1: WO 2021/182595

### SUMMARY

An information processing system according to one aspect of the present disclosure includes: an acquirer configured to acquire medical information including first medical information including a medical image showing a bone of a subject and second medical information including feature information of the subject at a time point different from a time point at which the medical image is captured; and an estimator configured to estimate bone state information on a future or past bone state of the subject by inputting the medical information into a learning model trained using training data including a plurality of pieces of information each including a medical image showing a bone of a predetermined person and feature information of the predetermined person at a time point different from a time point at which the medical image is captured.

A method for controlling an information processing system according to one aspect of the present disclosure includes: acquiring medical information including first medical information including a medical image showing a bone of a subject and second medical information including feature information of the subject at a time point different from a time point at which the medical image is captured; and estimating bone state information on a future or past bone state of the subject by inputting the medical information into a learning model trained using training data including a plurality of pieces of information each including a medical image showing a bone of a predetermined person and feature information of the predetermined person at a time point different from a time point at which the medical image is captured.

### BRIEF DESCRIPTION OF THE DRAWINGS

FIG. 1 is a diagram illustrating a configuration example of an information processing system according to one aspect of the present disclosure.

FIG. 2 is a block diagram illustrating an example of a configuration of an information processing device according to one aspect of the present disclosure.

FIG. 3 is a diagram illustrating an example of a configuration of a learning model executed by an estimator.

FIG. 4 is a flowchart illustrating an example of a flow of training processing by a trainer.

FIG. 5 is a flowchart illustrating an example of a flow of processing executed by the information processing device.

FIG. 6 is a diagram illustrating a configuration example of an information processing system according to another aspect of the present disclosure.

FIG. 7 is a flowchart illustrating an example of a flow of training processing by a trainer.

FIG. 8 is a flowchart illustrating an example of a flow of processing executed by the information processing device.

### DESCRIPTION OF EMBODIMENTS

Embodiments according to the present disclosure will be described below. Although a case in which a target whose future or past bone state information on a bone state is estimated is a human (that is, a “subject”) is described as an example in the following description, the target is not limited to a human. That is, the “subject” according to the present disclosure may be, for example, a mammal or a vertebrate other than a human, such as those from the equine, the feline, the canine, the bovine, or the porcine. The present disclosure also includes an embodiment in which the “subject” is reworded as “animal” when the embodiment out of the following embodiments is applicable to any of such animals.

### First Embodiment

An information processing device 1 according to one embodiment of the present disclosure is described in an example below in detail.

The information processing device 1 outputs estimated information of bone state information on a future or past bone state of a subject, and the estimated information is estimated using a learning model, based on input information including medical information of the subject. The medical information of the subject includes first medical information including a medical image showing a bone of the subject and second medical information including feature information of the subject at a time point different from a time point at which the medical image is captured. The learning model has been trained using training data including a plurality of pieces of information each including a medical image showing a bone of a predetermined person and feature information of the predetermined person at a time point different from a time point at which the medical image is captured.

### Configuration of Information Processing System 100a

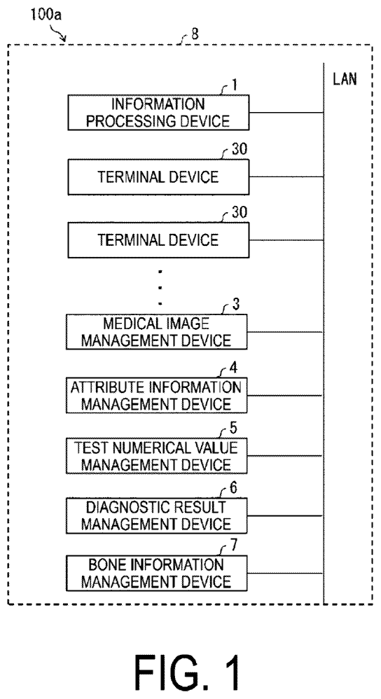

First, a configuration of an information processing system 100a according to one aspect of the present disclosure will be described with reference to FIG. 1. FIG. 1 is a diagram illustrating a configuration example of the information processing system 100a in a medical facility 8 in which the information processing device 1 is introduced.

The information processing system 100a includes the information processing device 1 and one or more terminal devices 30 communicably connected to the information processing device 1. The information processing device 1 estimates bone state information on a future or past bone state of a subject, from medical information that includes first medical information including a medical image showing a bone of the subject and second medical information including feature information of the subject at a time point different from a time point when the medical image is captured. In the following description, an example in which bone state information on a future bone state of a subject is estimated (predicted) will be described, and the “bone state information on a future bone state” will also be simply referred to as “future bone state information”. The information processing device 1 is a computer configured to transmit the estimated information of the future bone state information of the subject to the terminal device 30. The information processing device 1 may be a cloud-based device installed on the cloud. In such a case, the terminal device 30 transmits the first medical information to the information processing device 1 on the cloud via a communication network 9, and receives the estimated information estimated by the information processing device 1 via the communication network 9. The terminal device 30 may be an on-premises device installed in the medical facility 8 or a company that provides analysis services.

The terminal device 30 functions as an outputter in the information processing system 100a, and presents information received from the information processing device 1. The terminal device 30 is a computer used by medical personnel such as a doctor (medical personnel) belonging to the medical facility 8. The terminal device 30 is, for example, a personal computer, a tablet terminal, a smartphone, or the like. The terminal device 30 includes a communicator that transmits and receives data to and from another device, an inputter such as a keyboard and a microphone, a display capable of displaying information transmitted from the information processing device 1, and an outputter such as a speaker.

An example is illustrated in which a local area network (LAN) is installed and the information processing device 1 and the terminal devices 30 are connected to the LAN in the medical facility 8 illustrated in FIG. 1, but the present disclosure is not limited thereto. For example, the Internet, a telephone communication line network, an optical fiber communication network, a cable communication network, a satellite communication network, or the like may be applied to the network in the medical facility 8. The information processing system 100a can be adapted to a hospital information system (HIS), a radiology information system (RIS), a picture archiving and communication system (PACS) or the like in the medical facility 8. Communication in the information processing system 100a conforms to an international standard such as digital imaging and communications in medicine (DICOM).

In addition to the information processing device 1 and the terminal device 30, a medical image management device 3, an attribute information management device 4, a test numerical value management device 5, a diagnostic result management device 6, and a bone information management device 7 may be communicably connected to the LANs in the medical facility 8.

The medical image management device 3 is a computer functioning as a server for managing medical images taken in the medical facility 8. The medical image management device 3 may be installed, for example, in the medical facility 8 or may be the cloud. When the medical image management device 3 is the cloud, the medical image can be acquired via the communication network 9.

The medical image may be an image showing at least part of a bone. The medical image may be an image showing at least part of at least any of trabecular bone, cortical bone, and/or cancellous bone. The medical image may be an image not showing a bone. The medical image may be any one of an X-ray image, a computed tomography (CT) image, a magnetic resonance imaging (MRI) image, an ultrasound image captured by an ultrasound diagnostic apparatus, a positron emission tomography (PET) image, a dual-energy X-ray absorptiometry (DXA) image. The medical image may be, for example, an inspection apparatus image acquired from an inspection apparatus (for example, an X-ray inspection apparatus), or an image obtained by reducing noise of the captured inspection apparatus image. For the noise reduction, for example, machine learning using training data in which some of training images classified as having or not having noise can be used. The information processing device 1 may acquire a medical image showing a bone of the subject, from the medical image management device 3. The X-ray image may include a panoramic X-ray image, for example for use in dentistry. The panoramic X-ray image is, for example, an image including a plurality of teeth (for example, all the teeth).

Such a medical image may be at least one selected from the group consisting of a front image showing a target site from a front (for example, an image obtained by irradiating the target site with X-rays in a front-back direction) and a side image showing the target site from a side (for example, an image obtained by irradiating the target site with X-rays in a left-right direction). As the medical image, for example, a chest X-ray front image including a chest of a person or a waist X-ray front image including a waist of a person can be used. The chest X-ray image is, for example, an image showing at least one selected from the group consisting of ribs, clavicles, and a sternum. The waist X-ray image is, for example, an image showing at least one selected from the group consisting of the lumbar vertebra, the pelvis, and the femur. The medical image is not limited to the waist or the chest, and an image showing a tooth, a jaw, an arm, a hand, a shoulder joint, a knee joint, a heel, a skull, or a bone of a foot may be used for example.

When a CT image is used as a medical image, information on a trabecular bone based on a three dimensionally constructed image may be used, or information on a trabecular bone based on a two dimensionally captured image may be used. When using a CT image as a medical image, for example, at least one selected from the group consisting of a three-dimensional image, a cross-sectional image perpendicular to a body axis connecting the head and legs (e.g., horizontal section), and a cross-sectional image parallel to the body axis (such as, for example, sagittal section or coronal section) may be used.

The medical image may show any of a head, a neck, a chest, a lumbar region, a hip joint, a knee joint, an ankle joint, a foot, a toe, a shoulder joint, an elbow joint, a wrist joint, a hand, and a maniphalanx. The medical image may be a dental image.

The attribute information management device 4 is a computer functioning as a server for managing attribute information of the subject. The information processing device 1 may acquire the attribute information of the subject from the attribute information management device 4. When the attribute information is added to the acquired medical image, the information processing device 1 may extract the attribute information from the medical image. The attribute information includes at least one selected from the group consisting of an age, a sex, a height, a weight, a race, lifestyle habit information, medication information, occupation information, blood test information, urine test information, saliva test information, a medical history, a family medical history of the subject, genetic information, childbirth information, menopause information, items of fracture risk assessment tool (FRAX (trade name)), and menopause estimation estimated based on hormone information. The childbirth information includes at least one selected from the group consisting of the presence or absence of a childbirth, and the number of childbirths. The lifestyle habit may be, for example, a sleep time, a wake-up time, a sleep time, a daily exercise amount, a meal content, a meal time, a meal time period, a blood sugar level, and the like. The meal content includes, for example, at least one selected from the group consisting of a meal name, an ingested food material, and an intake amount. The meal content may be an estimated intake including at least one selected from the group consisting of, for example, calcium, vitamin B, vitamin D, and vitamin K. For example, a designated value estimated from a parameter acquired by a wearable device may be employed for the blood glucose level.

The medication information may include information such as a name of a medication, an amount of the medication taken, and a period of taking the medication for example. The information on the medication being taken may include information on a steroid agent being used. The blood test information may be information on the result of at least any of, for example, a biochemical test, a glucose metabolism test, and an endocrine test.

The test numerical value management device 5 is a computer functioning as a server for managing a test numerical value obtained by a test taken in the medical facility 8. The test numerical value includes at least one selected from the group consisting of the bone density, Kellgren-Lawrence (KL) classification, a bone morphology angle, a muscle mass, mini mental state examination (MMSE), a blood test numerical value (for example, at least one selected from the group consisting of bone formation markers, bone resorption markers, and vitamin K levels), a hepatic function marker, a uric acid level, and a malignant tumor marker. The bone evaluation information may include information assessed by a fracture risk assessment tool. The information processing device 1 may acquire the test numerical value of the subject from the test numerical value management device 5.

The diagnostic result management device 6 is a computer functioning as a server for managing diagnostic results from a diagnosis in the medical facility 8. The information processing device 1 may acquire the diagnostic result of the subject from the diagnostic result management device 6. The diagnostic result may include at least one selected from the group consisting of the presence or absence of a bone fracture and the degree of osteoporosis. The fracture may be accompanied by information on the cause, such as a fragility fracture, a fatigue fracture, or a traumatic fracture for example.

The bone information management device 7 is a computer functioning as a server for managing bone state information acquired in the medical facility 8. The bone state information may include, for example, at least one selected from the group consisting of the presence or absence of a fracture, a possibility of osteoporosis, a drug efficacy, an incident occurrence, a bone volume, a bone density, a trabecular number, a trabecular separation, a trabecular thickness, a trabecular orientation, a trabecular connectivity density, and a trabecular bone score. The bone state information may be obtained by analyzing the strength of the bone from the state of the cortical bone and/or the cancellous bone. The information processing device 1 may acquire the past bone state information of the subject from the bone information management device 7.

The possibility of osteoporosis may be, for example, a result of classification based on at least one selected from the group consisting of the presence or absence of a fracture, the possibility of a fracture, and a change in bone density. The possibility of osteoporosis includes “no osteoporosis”, “suspected osteoporosis”, or “osteoporosis”. More specifically, when no disease leading to a bone volume reduction is present, no secondary osteoporosis is observed, and a fracture or a high possibility of fracture is present, the possibility of osteoporosis may be a classification result indicating classification as primary osteoporosis. The drug efficacy may include, for example, a name of a drug that improves the bone condition when taken and/or administered for a certain period of time, or may include information indicating at least one selected from the group consisting of the bone density, the bone volume, and the trabecular bone condition after a certain period of time has elapsed. Examples of the occurrence of an incident may include, for example, loosening of an implant, removal of an implant, or an infection around an implant. Examples of the implant include an artificial hip joint, an artificial knee joint, a spinal implant, a bolt to be embedded in a bone, or a dental implant.

The LAN in the medical facility 8 may be communicably connected to an external communication network. In the medical facility 8, the information processing device 1 and the terminal device 30 may be directly connected to each other without going through the LAN.

### Configuration of Information Processing Device 1

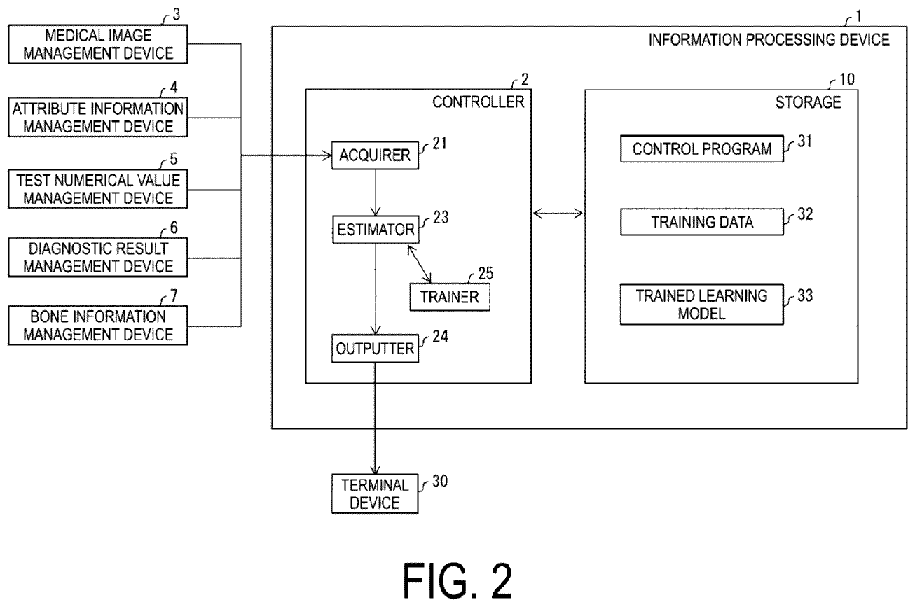

A configuration of the information processing device 1 applied to the information processing system 100a illustrated in FIG. 1 is described with reference to FIG. 2. FIG. 2 is a block diagram illustrating an example of a configuration example of the information processing device 1.

The information processing device 1 includes a controller 2 that integrally controls each component of the information processing device 1, and a storage 10 that stores various types of data to be used by the controller 2. The controller 2 includes an acquirer 21, an estimator 23, an outputter 24, and a trainer 25. The storage 10 stores training data 32 and a trained learning model 33 in addition to a control program 31, which is a program for executing various types of controls of the information processing device 1.

### Acquirer 21

The acquirer 21 acquires input information including medical information of a subject. The input information is data input to the estimator 23. The input information includes the first medical information including a medical image showing a bone of the subject and the second medical information including feature information of the subject at a time point different from a time point at which the medical image is captured. In the following description, a medical image showing a bone of the subject and being included in the first medical information may be referred to as a first medical image. The second medical information may be information at a time point when a period of, for example, 15 days, one month, three months, six months, one year, three years, or five years has elapsed from a time point when the medical image of the first medical information is captured. The second medical information may be past information or future information before or after the time point at which the medical image included in the first medical information is captured.

The feature information included in the second medical information includes at least one selected from the group consisting of a medical image of the subject, past bone state information, attribute information, a test numerical value, and a diagnostic result. In the following description, a medical image showing a bone of the subject and being included in the second medical information may be referred to as a second medical image. The second medical information may be information at a plurality of time points or at one time point.

For example, the acquirer 21 may acquire the first medical image and/or the second medical image from the medical image management device 3, attribute information of the subject from the attribute information management device 4, a test numerical value of the subject from the test numerical value management device 5, a diagnostic result of the subject from the diagnostic result management device 6, or past bone state information of the subject from the bone information management device 7. For example, the acquirer 21 acquires, as input information, an X-ray image captured at a first time point as the first medical information and an X-ray image captured at a time point different from the first time point, for example, at a second time point before the first time point as the second medical information. The acquirer 21 may further acquire, as the input information, an X-ray image captured at a third time point before the second time point as the second medical information.

The first medical information may include the same information element as the feature information included in the second medical information. In other words, the first medical information may include the same information as the feature information included in the second medical information, in addition to the first medical image. For example, when the second medical information includes the height information of the subject as the feature information, the first medical information may include, in addition to the first medical image, the height information of the subject at a time point when the first medical image is captured.

### Estimator 23

The estimator 23 estimates future bone state information of the subject by inputting the input information acquired by the acquirer 21 to the learning model 33. Here, the learning model 33 is trained in advance using the training data 32. The training data 32 is data including medical information of a predetermined person. The predetermined person may be a patient suffering from a bone-related disease or a person not suffering from a disease. The medical information included in the training data 32 may include a plurality of pieces of information including a medical image showing a bone of a predetermined person and feature information of the predetermined person at a time point different from a time point at which the medical image is captured.

The estimator 23 may estimate future or past bone state information of the subject by inputting, to the learning model 33, as the input information, input information including the first medical information including a medical image showing a bone of the subject and the second medical information including feature information of the subject at a time point different from a time point at which the medical image is captured. The estimator 23 may output information such as a value related to the training data 32 used for training the learning model 33. By inputting the input information to the learning model 33, the estimator 23 may estimate, as the bone state information, at least one selected from the group consisting of the future or past occurrence probability of fracture of the subject, a time point when a future or past fracture will occur or has occurred in the subject (such as, for example, four to five years later), a future or past bone density of the subject, and a future or past bone quality of the subject. More specifically, when the bone state information is the bone density, the estimator 23 can estimate how bone density was when the past first medical information was acquired, for example, even if the bone density was not measured in the past. The estimator 23 may estimate the bone density of the subject at a plurality of future and past time points from the first medical information. The estimator 23 may estimate the transition of the bone density of the subject from a certain time point in the past to a certain time point in the future. The information processing device 1 may graphically display the estimated transition on the display.

A measured value obtained by actually measuring the bone density from at least one selected from the group consisting of, for example, a hand, a lumbar spine, a proximal femur, a tibia, a heel, and an arm (such as, for example, a radius) can be employed for the bone density. For example, a single energy X-ray absorptiometry method, a dual-energy X-ray absorptiometry method, an ultrasound method, a micro densitometry (MD) method, or a quantitative computed tomography (CT) method can be employed for the measurement of the bone density. In a DXA apparatus that measures a bone density using the DXA method, when the bone density of the lumbar vertebrae is measured, X-rays are emitted to the lumbar vertebrae of a human subject from the front of the lumbar vertebrae. In the DXA apparatus, when the bone density of the proximal femur is measured, X-rays are emitted to the proximal femur of the human subject from the front of the proximal femur. Here, the “front of the lumbar vertebrae” and the “front of the proximal femur” are intended to be directions correctly facing capturing sites such as the lumbar vertebrae and the proximal femur, and may be the ventral side of the human subject's body or the back side of the human subject. The proximal femur includes, for example, at least one site selected from the group consisting of the neck, the trochanter, the diaphysis, and the entire proximal femur (neck, trochanter, and diaphysis). In the MD method, for example, a hand is irradiated with X-rays.

The bone density may be a value related to the density of bone. A bone mineral density may be represented by at least one type of bone density per unit area (g/cm2), bone density per unit volume (g/cm³), YAM (%), T-score, and Z-score. YAM (%) is an abbreviation for “young adult mean” and is sometimes called a young adult average percentage. The bone density of the bone may be an index determined by a guideline or may be a unique index. The bone density can be, for example, a value used in osteoporosis guidelines (such as, but not limited to, 2015 Prevention and Treatment Guidelines of the Japan Osteoporosis Society, General Incorporated Association).

The estimator 23 may estimate, as the bone state information, what bone state information will be achieved or how bone state information will change, as a consequence of treatment for the subject. More specifically, when the bone state information is the future bone density of the subject, the estimator 23 may estimate, as the bone state information, the bone density or a change in the bone density that will be achieved as a consequence of treatment for the subject. The estimator 23 may estimate the content of the treatment in addition to the bone state information. The information processing device 1 may display the estimated treatment content on the display. The treatment can include, for example, taking medications, taking nutrients, or improving a lifestyle habit, for a predetermined period. The medication may include information on a medication having at least any of an effect on bone formation and an effect on bone resorption. Examples of the medication having an effect on bone formation may include, but are not limited to, an activated vitamin D3 preparation (for example, Calcitriol, Eldecalcitol, or Alfacalcidol), Teriparatide Acetate, and Teriparatide (recombinant). Examples of the medication having an effect on bone resorption may include, but are not limited to, a calcitonin preparation, a bisphosphonate preparation, and an anti-RANKL monoclonal antibody.

The learning model 33 is an operation model used by the estimator 23 in executing operations, based on the input data. The learning model 33 is generated by the trainer 25 executing machine learning using the training data 32, which will be described below, on an untrained neural network. Here, the learning model 33 may also be applied to an animal other than a human. In such a case, the “predetermined person” in the training data 32 may be a biological species of the same type as the “subject”. That is, the information processing device 1 according to the present disclosure can also estimate the onset and/or progression of diseases of an animal other than a human. A specific example of the training data 32, a configuration of the neural network, and the training processing will be described below.

### Outputter 24

The outputter 24 transmits information estimated by the estimator 23 to the terminal device 30. The information processing device 1 may include a display (not illustrated). In such a case, the outputter 24 causes the display to display the information estimated by the estimator 23.

### Trainer 25

The trainer 25 controls training processing for the untrained neural network. The trainer 25 creates a trained neural network functioning as the estimator 23 by executing training processing on an untrained neural network. The training data 32 (described below) is used for such training. A specific example of training executed by the trainer 25 will be described below.

### Configuration of Estimator 23

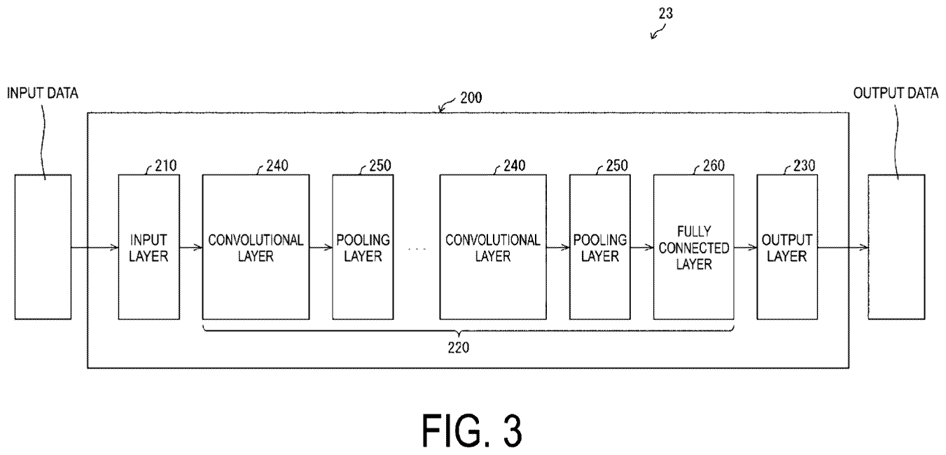

A configuration of the estimator 23 will be described below with reference to FIG. 3. The configuration illustrated in FIG. 3 is merely an example and the configuration of the estimator 23 is not limited thereto.

As illustrated in FIG. 3, the estimator 23 performs operations based on the learning model 33 on the input data input to the input layer 210 to output output data from the output layer 230. The output data is future bone state information of the subject. The future bone state information may be, for example, a future occurrence probability of fracture of the subject, a future bone density of the subject, or a future bone quality of the subject.

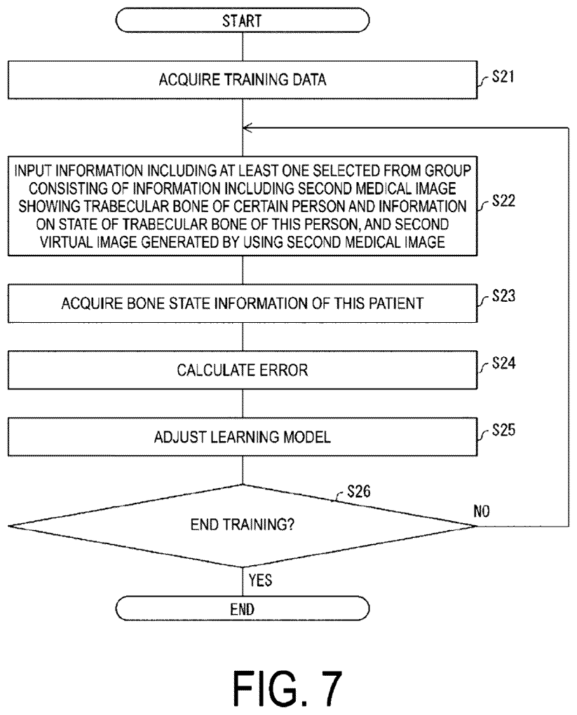

The estimator 23 of FIG. 3 includes a neural network 200 including the input layer 210 and the output layer 230. FIG. 3 illustrates a case where the neural network 200 is a convolutional neural network (CNN). As illustrated in FIG. 3, the neural network 200 includes, for example, an input layer 210, a hidden layer 220, and an output layer 230. The hidden layer 220 is also referred to as an intermediate layer. The hidden layer 220 includes, for example, a plurality of convolutional layers 240, a plurality of pooling layers 250, and a fully connected layer 260. In the neural network 200, the fully connected layer 260 is present in a stage preceding the output layer 230. In the neural network 200, the convolutional layers 240 and the pooling layers 250 are alternately arranged between the input layer 210 and the fully connected layer 260. The configuration of the neural network 200 is not limited to the example in FIG. 7. For example, the neural network 200 may include one convolutional layer 240 and one pooling layer 250 between the input layer 210 and the fully connected layer 260.

The neural network 200 is not limited to the CNN, and may be an LSTM. For example, the neural network 200 may be a ConvLSTM network or the like in which CNN and LSTM are combined. In this case, the input layer 210 can extract a feature amount for a change in the input data. The output layer 230 can calculate a new feature amount, based on the feature amount extracted in the input layer 210 and the temporal change and the initial value of the input data. Each of the input layer 210 and the output layer 230 includes a plurality of LSTM layers. Each of the input layer 210 and the output layer 230 may include three or more LSTM layers.

### Training Processing by Trainer 25

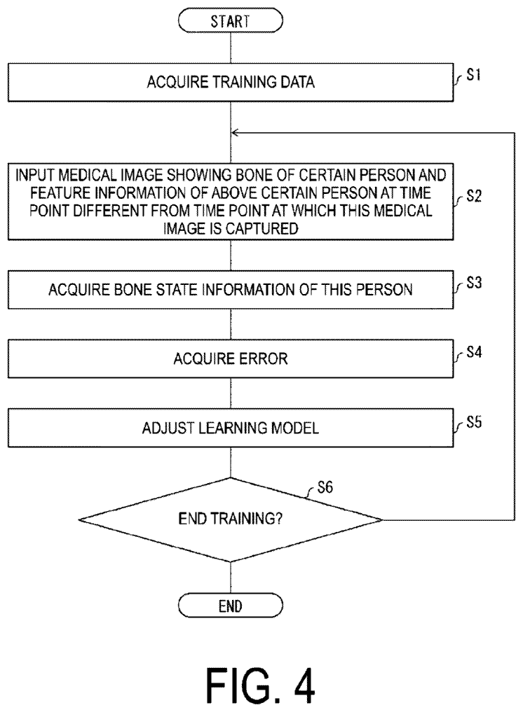

The training processing for generating the learning model 33 will be described below with reference to FIG. 4. FIG. 4 is a flowchart illustrating an example of a flow of training processing by the trainer 25.

The trainer 25 acquires the training data 32 from the storage 10 (step S1). The training data 32 includes a plurality of pieces of information including bone state information on a future bone state of a predetermined person, and includes an explanatory variable and an objective variable to be input to the input layer 210. Here, the explanatory variable is a medical image showing a bone of a predetermined person and feature information of the predetermined person at a time point different from a time point at which the medical image is captured, and the objective variable is bone state information of each person. The training data 32 may include content of treatment of a predetermined person. The treatment can include, for example, taking medications, taking nutrients, or improving a lifestyle habit, for a predetermined period. The medication may include information on a medication having at least any of an effect on bone formation and an effect on bone resorption. Examples of the medication having an effect on bone formation may include, but are not limited to, an activated vitamin D3 preparation (for example, Calcitriol, Eldecalcitol, or Alfacalcidol), Teriparatide Acetate, and Teriparatide (recombinant). Examples of the medication having an effect on bone resorption may include, but are not limited to, a calcitonin preparation, a bisphosphonate preparation, and an anti-RANKL monoclonal antibody.

The trainer 25 inputs a medical image showing a bone of a certain person (referred to as a person A) and feature information of the person A at a time point different from a time point at which the medical image is captured, to the input layer 210 as information including future bone state information (step S2).

The trainer 25 acquires output data regarding the bone state information of the person A from the output layer 230 (step S 3). The output data contains the same contents as the objective variable of the training data 32. In FIG. 4, the order of step S2 and step S3 may be reversed. Alternatively, in FIG. 4, step S2 and step S3 may be executed simultaneously.

The trainer 25 acquires an objective variable for the person A included in the training data 32. The trainer 25 compares the output data acquired in step S3 with the objective variable for the person A, calculates an error (step S4), and adjusts the learning model 33 being trained so as to reduce the error (step S5).

Any known method is applicable to the adjustment of the learning model being trained. For example, an error backpropagation method may be employed as a method for adjusting the learning model. The adjusted learning model becomes a new learning model, and in subsequent operations, the estimator 23 uses the new learning model. In the adjustment stage of the learning model, the parameters (such as, for example, a filter coefficient and a weighting factor) used in the estimator 23 may be adjusted.

If the error is not within the predetermined range, and if the explanatory variables for all the persons contained in the training data 32 are not input (NO in step S6), the trainer 25 returns to step S2 and repeats the training processing. If the error is within the predetermined range, and if the explanatory variables for all the persons contained in the training data 32 are input (YES in step S6), the trainer 25 ends the training processing.

When the training processing as described above is adopted, the estimator 23 can estimate the future bone state information of the subject from medical information that includes the first medical information including a medical image showing a bone of the subject and the second medical information including feature information of the subject at a time point different from a time point at which the medical image is captured.

In one aspect of the present disclosure, the learning model 33 may be a learning model trained using training data including medical information of the predetermined person acquired at a plurality of different time points. For example, the learning model 33 may be a learning model trained using training data including medical images of a predetermined person acquired at a plurality of different time points as an explanatory variable and including the bone density of the predetermined person acquired at the plurality of different time points as an objective variable. Accordingly, the estimation accuracy may be improved by inputting the medical information of the subject acquired at at least two time points different from each other to the learning model 33.

### Processing Executed by Information Processing Device 1

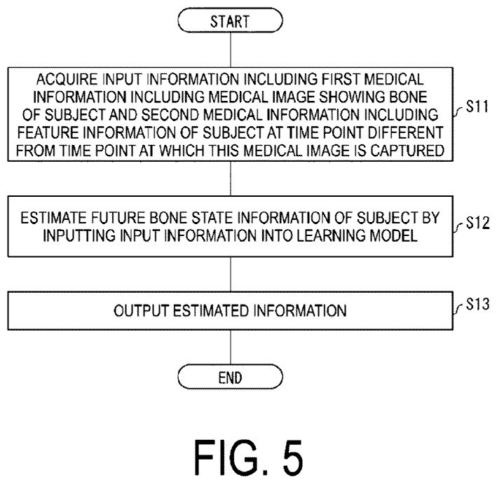

A flow of processing executed by the information processing device 1 will be described below with reference to FIG. 5. FIG. 5 is a flowchart illustrating an example of a flow of processing executed by the information processing device 1.

First, the acquirer 21 acquires medical information that includes the first medical information including a medical image showing a bone of a subject and the second medical information including feature information of the subject at a time point different from a time point at which the medical image is captured (step S11: acquiring step).

The estimator 23 inputs the acquired medical information to the learning model 33 to estimate future bone state information of the subject (step S12: estimating step).

The outputter 24 outputs each piece of information including the bone state information estimated in step S12 (step S13: outputting step).

According to such a configuration, the information processing device 1 and the information processing system 100a estimate the future bone state information of the subject from the medical information that includes the first medical information including the medical image showing a bone of the subject and the second medical information including the feature information of the subject at a time point different from a time point at which the medical image is captured. Accordingly, the estimation accuracy may be improved as compared with a case where the bone state information is estimated using the input information including the medical information acquired at one time point.

The estimator 23 may estimate the bone state information of the subject after a time shorter than a time interval between any two time points of the time point at which the first medical information is acquired and at least one time point at which the second medical information is acquired. Accordingly, the bone state information may be more accurately estimated based on the change information of the state of the subject in a time interval longer than the time interval from the current time point to the estimation time point.

The estimator 23 may estimate the future bone state information of the subject by inputting, to the learning model 33, input information that includes medical information at three or more time points, that is, the first medical information including a medical image showing a bone of the subject and the second medical information including feature information of the subject at two or more time points different from the time point at which the medical image is captured. Accordingly, the acceleration degree of the temporal change of the bone state up to the current time point can be known, and the bone state information at the estimation time point can be estimated with higher accuracy.

The estimator 23 may estimate whether the subject is menopausal from the attribute information of the subject and the degree of acceleration of the temporal change in the bone state at three or more time points. In such a case, the estimator 23 may use the estimated menopause information as the menopause information of the attribute information of the subject.

If the second medical information is a medical image, a site shown in the medical image of the second medical information may be different from a site shown in the medical image of the first medical information. For example, the site shown in the image of the first medical information may be the chest, and the medical image of the second medical information may be a dental medical image. As a result, images captured in a medical examination or a dental treatment can be flexibly used, and versatility is improved.

The estimator 23 may estimate the bone state information of the subject at a time (the time point is referred to as an estimation time point) after a time longer than a time interval (hereinafter referred to as a first time interval) between the earliest time point and the latest time point among the time point at which the first medical information is acquired and at least one time point at which the second medical information is acquired. As described above, the information processing system 100a according to the present disclosure estimates the future bone state information of the subject using the first medical information and the second medical information acquired at different time points, and thus, the future bone state information of the subject within the first time interval from the current time point may be estimated with high accuracy. Therefore, in estimating the bone state information of the subject after the first time interval from the current time point, the bone state information can be estimated using the future bone state information of the subject by the first time interval from the current time point estimated with high accuracy. As a result, the bone state information of the subject may be more accurately estimated compared to a case where the bone state information is estimated using medical information at one time point.

When the second medical information acquired by the acquirer 21 includes medical information at two or more time points and data of any of the second medical information at the two or more time points and the first medical information is anomalous data, the estimator 23 may input data excluding the anomalous data to the learning model 33. The anomalous data is, for example, a medically impossible test numerical value caused by a test error or the like. According to such a configuration, the possibility that erroneous information is input to the learning model 33 may be reduced to improve the estimation accuracy. In such a case, for example, the estimator 23 may identify, as thresholds, a lower limit value and an upper limit value of values that are medically possible as test numerical values, and may input, to the learning model 33, data excluding data outside the range of the thresholds.

When the second medical information acquired by the acquirer 21 includes medical information at two or more time points and data of any of the second medical information at the two or more time points and the first medical information is anomalous data, the estimator 23 may replace the anomalous data with data obtained by approximating the anomalous data to other data to be input into the learning model 33. According to such a configuration, the possibility that erroneous information is input to the learning model 33 may be reduced to improve the estimation accuracy.

Menopause is known to result in significant reduction of bone density. Therefore, when the subject is a woman who is not menopausal at the current time point, the estimator 23 may output at least one selected from the group consisting of a first estimation result on the assumption that the subject is menopausal and a second estimation result on the assumption that the subject is not menopausal, using menopause information included in the attribute information. Accordingly, by taking into account both cases, that is, menopausal or not menopausal, an estimation result with higher accuracy may be output. When the subject is a woman who is not menopausal at the current time point, the estimator 23 may simultaneously output the first estimation result on the assumption that the subject is menopausal and the second estimation result on the assumption that the subject is not menopausal.

It is known that a birth experience greatly affects bone density. Therefore, when the subject is a woman without birth experience, the estimator 23 may output at least one selected from the group consisting of the first estimation result on the assumption that the subject has birth experience and the second estimation result on the assumption that the subject has no birth experience, using birth information included in the attribute information. Accordingly, by taking into account birth experience, an estimation result with higher accuracy may be output.

### Second Embodiment

### Configuration of Information Processing System 100b

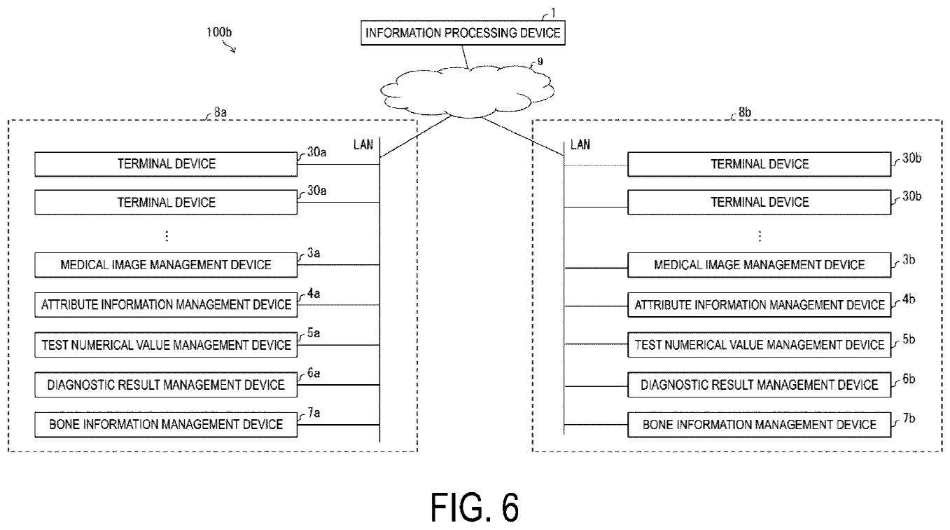

The information processing device 1 may be communicably connected to LANs each installed in one of a plurality of medical facilities 8 via a communication network 9, instead of a computer installed in the predetermined medical facility 8. FIG. 6 is a diagram illustrating a configuration example of the information processing system 100b according to another aspect of the present disclosure. For convenience of description, members having the same functions as those of the members described in the above-described embodiment are denoted by the same reference signs, and description thereof is not repeated.

In addition to the one or more terminal device 30a, the medical image management device 3a, an attribute information management device 4a, a test numerical value management device 5a, a diagnostic result management device 6a, and a bone information management device 7a may be communicably connected to the LAN in a medical facility 8a. In addition to the terminal device 30b, the medical image management device 3b, an attribute information management device 4b, a test numerical value management device 5b, a diagnostic result management device 6b, and a bone information management device 7b may be communicably connected to the LAN in a medical facility 8b. In the present disclosure, when no particular distinction is made between the medical facilities 8a and 8b, each of the medical facilities 8a and 8b is referred to as a “medical facility 8”. When no particular distinction is made between the terminal devices 30a and 7b, between the medical image management devices 3a and 3b, between the attribute information management devices 4a and 4b, between the test numerical value management devices 5a and 5b, between the diagnostic result management devices 6a and 6b, and between the bone information management devices 7a and 7b, each of them is also referred to as the “terminal device 30”, the “medical image management device 3”, the “attribute information management device 4”, the “test numerical value management device 5”, the “diagnostic result management device 6”, and the “bone information management device 7”, respectively.

FIG. 6 illustrates an example in which the LANs in the medical facility 8a and the medical facility 8b are connected to the communication network 9. The information processing device 1 is not limited to the configuration illustrated in FIG. 6 as long as the information processing device 1 is communicably connected to the medical image management device 3, the attribute information management device 4, the test numerical value management device 5, the diagnostic result management device 6, and the bone information management device 7 in each medical facility via the communication network 9. For example, the information processing device 1 may be installed in the medical facility 8a or in the medical facility 8b.

In the information processing system 100b employing such a configuration, the information processing device 1 can acquire a medical image of a subject Pa examined in the medical facility 8a, the attribute information, the test numerical value, and the diagnostic result, from the medical image management device 3a, the attribute information management device 4a, the test numerical value management device 5a, and the diagnostic result management device 6a in the medical facility 8a, respectively. The information processing device 1 transmits the estimation result of the future bone state information of the subject Pa to the terminal device 30a installed in the medical facility 8a. The information processing device 1 transmits the future bone state information of a subject Pb to the terminal device 30b.

### Third Embodiment

A configuration may be employed that outputs estimation information of bone state information on a state of a bone of a subject, which is estimated using the learning model 33 as in the information processing device 1, also based on input information including third medical information different from the first medical information and the second medical information. In this case, the third medical information of the subject includes at least one selected from the group consisting of the first medical image showing the trabecular bone of the subject and a first virtual image in which the color shade in the first medical image is expressed for each predetermined area (pixel). The learning model 33 may be trained using training data including at least one selected from the group consisting of information including the second medical image showing a trabecular bone of a predetermined person and trabecular bone information on a state of the trabecular bone of the predetermined person, and a second virtual image in which a color shade in the second medical image is expressed for each predetermined area. The first medical image and the second medical image may be images that are the same as or similar to the medical images described above.

The information processing system 100a having such a configuration will be described below. For convenience of description, members having the same functions as those of the members described in the above-described embodiment are denoted by the same reference signs, and description thereof is not repeated.

### Configuration of Information Processing System 100a

The information processing system 100a according to the present embodiment includes the information processing device 1 and one or more terminal devices 30 communicably connected to the information processing device 1 as illustrated in FIG. 1. The information processing device 1 estimates bone state information on a state of a bone of a subject from medical information including at least one selected from the group consisting of a medical image showing a trabecular bone of the subject and the first virtual image in which a color shade in the medical image is expressed for each predetermined area. The information processing device 1 may estimate future bone state information of the subject or may estimate current bone state information of the subject. The information processing device 1 is a computer configured to transmit the estimated information of the bone state information of the subject to the terminal device 30.

The medical image management device 3 generates a virtual image in which the color shade of the medical image is expressed for each predetermined area. The virtual image is generated from the density of the cancellous bone and/or cortical bone portion and the density of the portion other than the bone in the bone image information including the bone portion in the medical image. More specifically, the virtual image can be generated as an image in which the density of the bone is digitized for each predetermined area from the bone image information and the numerical value is expressed by the color shade, for example.

One unit of the predetermined area may be, for example, one pixel of the image or a plurality of pixels of the image. In the predetermined area, for example, at least one selected from the group consisting of the trabecular bone, the trabecular number (such as, for example, the number of trabecular bones present per unit length), the trabecular separation (for example, the spatial distance between trabecular bones), the trabecular thickness (such as, for example, the width or thickness of the trabecular bone), the trabecular orientation (such as, for example, the degree of alignment of the trabecular directions in the beam), and the connectivity density of the bone (for example, the number of paths connecting the ends of the trabecular bones per unit area) may be taken into consideration. As the numerical value, for example, at least one selected from the group consisting of a bone volume (such as, for example, a numerical value in units of g), a bone density, and a trabecular bone score can be used. For example, two gradations of white and black, a gray scale (for example, a color obtained by adding a color in which the density of gray, which is an intermediate color between white and black, is expressed in 254 gradations to two gradations of white and black), a heat map using a plurality of colors, or the like can be used as the color shade. The color shade is not limited to these, and for example, white as one of two gradations may be displayed transparently, or a predetermined numerical value or more may be a target of the shade. The information processing device 1 may acquire a medical image showing a trabecular bone of the subject and a virtual image generated using the medical image, from the medical image management device 3.

Although FIG. 1 illustrates a configuration example including the medical image management device 3, the attribute information management device 4, the test numerical value management device 5, the diagnostic result management device 6, and the bone information management device 7, this is not to be construed in a limiting sense. For example, a management device having the functions of some or all of the plurality of devices may be provided.

### Configuration of Information Processing Device 1

A configuration of the information processing device 1 applied to the information processing system 100a according to the present embodiment is described with reference to FIG. 2.

### Acquirer 21

The acquirer 21 acquires input information including medical information of a subject. The input information is data input to the estimator 23. The input information includes at least one selected from the group consisting of a medical image showing a trabecular bone of the subject and a virtual image in which a color shade in the medical image is expressed for each predetermined area. In the following description, the medical image showing the trabecular bone of the subject may be referred to as a first medical image, and the virtual image in which the color shade in the medical image is expressed for each predetermined area may be referred to as a first virtual image. The acquirer 21 may acquire the first medical image and/or the first virtual image from the medical image management device 3. The acquirer 21 may acquire, from a virtual image generation device other than the medical image management device 3, the first virtual image generated by the virtual image generation device using the first medical image. The acquirer 21 may acquire as the input information, attribute information of the subject from the attribute information management device 4, a test numerical value of the subject from the test numerical value management device 5, a diagnostic result of the subject from the diagnostic result management device 6, or past bone state information of the subject from the bone information management device 7 as input information, in addition to the first medical image and the first virtual image.

### Estimator 23

The estimator 23 estimates bone state information of the subject by inputting the input information acquired by the acquirer 21 to the learning model 33. In the present embodiment, an example in which the estimator 23 estimates future bone state information of the subject will be described, but past bone state information of the subject may be estimated. Here, the learning model 33 is trained in advance using the training data 32. The training data 32 is data including medical information of a predetermined person. The predetermined person may be a patient suffering from a bone-related disease or a person not suffering from a disease. The medical information included in the training data 32 includes at least one selected from the group consisting of information including the second medical image showing a trabecular bone of a predetermined person and trabecular bone information on a state of the trabecular bone of the predetermined person, and the second virtual image in which a color shade in the second medical image is expressed for each predetermined area. In the following description, the medical image showing the trabecular bone of the predetermined person may be referred to as the second medical image, and the virtual image in which the color shade in the second medical image is expressed for each predetermined area may be referred to as a second virtual image.

The estimator 23 may estimate the future bone state information of the subject by inputting input information including at least one selected from the group consisting of the first medical image and the first virtual image into the learning model 33. The estimator 23 may estimate the future bone state information of the subject by (1) inputting the medical information including the first medical image into the learning model 33 trained using the training data including at least the information including the second medical image showing the trabecular bone of the predetermined person and the trabecular bone information related to the state of the trabecular bone of the predetermined person, or (2) inputting the medical information including the first virtual image into the learning model 33 trained using the training data including at least the second virtual image in which the color shade in the second medical image is expressed for each predetermined area. The estimator 23 may estimate at least one selected from the group consisting of a trabecular bone score, an occurrence probability of fracture, a possibility of osteoporosis, a drug efficacy, an incident occurrence, a bone density, a trabecular number, a trabecular separation, a trabecular thickness, a trabecular orientation, and a connectivity density of bone as the bone state information by inputting the input information to the learning model 33. The bone state information may be obtained by analyzing the strength of the bone from the state of the cortical bone and/or the cancellous bone.

### Configuration of Estimator 23

As illustrated in FIG. 3, the estimator 23 performs operations based on the learning model 33 on the input data input to the input layer 210 to output output data from the output layer 230. The output data is future bone state information of the subject. The future bone state information may be, for example, at least one selected from the group consisting of a trabecular bone score, an occurrence probability of fracture, a possibility of osteoporosis, a drug efficacy, an incident occurrence, a bone density, a trabecular number, a trabecular separation, a trabecular thickness, a trabecular orientation, and a connectivity density of bone.

### Training Processing by Trainer 25

The training processing for generating the learning model 33 will be described below with reference to FIG. 7. FIG. 7 is a flowchart illustrating an example of a flow of training processing by the trainer 25.

The trainer 25 acquires the training data 32 from the storage 10 (step S 21). The training data 32 includes at least one selected from the group consisting of information including the second medical image showing a trabecular bone of a predetermined person and trabecular bone information on a state of the trabecular bone of the predetermined person and the second virtual image, and data related to a state of the trabecular bone of the predetermined person after a predetermined time from capturing of the second medical image, and includes an explanatory variable and an objective variable to be input into the input layer 210. Here, the explanatory variable is at least one selected from the group consisting of information including the second medical image showing a trabecular bone of a predetermined person and trabecular bone information on a state of the trabecular bone of the predetermined person, and the second virtual image, and the objective variable is data related to a state of the trabecular bone of the predetermined person after a predetermined time from capturing of the second medical image.

Subsequently, the trainer 25 inputs, into the input layer 210, information including at least one selected from the group consisting of information including the second medical image showing a trabecular bone of a certain person (referred to as a person A) and trabecular bone information on a state of the trabecular bone of the predetermined person and the second virtual image (step S22).

The trainer 25 acquires output data regarding the bone state information of the person A from the output layer 230 (step S23). The output data contains the same contents as the objective variable of the training data 32. In FIG. 7, the order of step S22 and step S23 may be reversed. Alternatively, in FIG. 7, step S22 and step S23 may be executed simultaneously.

The trainer 25 acquires an objective variable for the person A included in the training data 32. The trainer 25 compares the output data acquired in step S23 with the objective variable for the person A, calculates an error (step S24), and adjusts the learning model being trained so as to reduce the error (step S25).

If the error is not within the predetermined range, and if the explanatory variables for all the persons contained in the training data 32 are not input (NO in step S26), the trainer 25 returns to step S2 and repeats the training processing. If the error is within the predetermined range, and if the explanatory variables for all the persons contained in the training data 32 are input (YES in step S26), the trainer 25 ends the training processing.

When the training processing as described above is employed, the estimator 23 can estimate the future bone state information of the subject from the medical information including at least one selected from the group consisting of the first medical image showing the trabecular bone of the subject and the first virtual image in which the color shade in the first medical image is expressed for each predetermined area. When the training data 32 includes a plurality of first medical images and/or first virtual images of the same person, the learning model 33 may be configured based on a temporal change in the feature amount of the characteristic region. Accordingly, the future bone state information of the subject can be estimated in consideration of the temporal change in the condition of the trabecular bone, and thus the accuracy of the estimation can be improved.

### Processing Executed by Information Processing Device 1

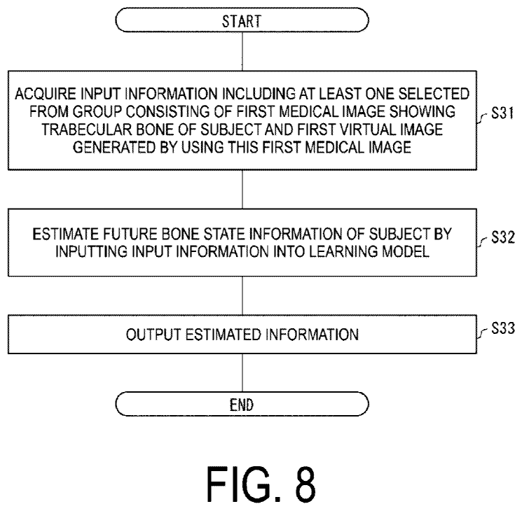

A flow of processing executed by the information processing device 1 will be described below with reference to FIG. 8. FIG. 8 is a flowchart illustrating an example of a flow of processing executed by the information processing device 1.

First of all, the acquirer 21 acquires medical information including at least one selected from the group consisting of the first medical image showing a trabecular bone of a subject and the first virtual image in which a color shade in the first medical image is expressed for each predetermined area (step S31: obtaining step).

The estimator 23 inputs the acquired medical information to the learning model 33 to estimate future bone state information of the subject (step S32: estimating step).

The outputter 24 outputs each piece of information including the bone state information estimated in step S32 (step S33: outputting step).

According to this configuration, the information processing device 1 and the information processing system 100a estimate the future bone state information of the subject from the medical information including at least one selected from the group consisting of the first medical image showing the trabecular bone of the subject and the first virtual image in which the color shade in the first medical image is expressed for each predetermined area. Here, the first medical image shows a trabecular bone which is an internal structure of a bone. Therefore, the estimation result output from the learning model 33 by the estimator 23 using the first medical image or the first virtual image generated using the first medical image as input information reflects the internal structure of the bone, and thus the estimation accuracy is high. That is, the information processing device 1 and the information processing system 100a can estimate the bone state information on the future state of the bones of the subject with high accuracy.

In the above description, the information processing device 1 estimates bone state information regarding a future bone condition of a subject, but the information processing device 1 of the present disclosure is not limited thereto. The information processing device 1 may estimate bone state information on the current condition of the bone of the subject. In this case, the trainer 25 may generate the learning model 33 using data related to the state of the trabecular bone of the predetermined person at the time point of capturing the second medical image as an objective variable. Accordingly, the estimator 23 can estimate the bone state information on the current state of the bone of the subject by inputting medical information including at least one selected from the group consisting of the first medical image and the first virtual image into the learning model 33.

Menopause is known to result in significant reduction of bone state. Therefore, when the bone state information on the future state of the bone of the subject who is a woman who is not menopausal at the current time point, the estimator 23 may output at least one selected from the group consisting of the first estimation result on the assumption that the subject is menopausal and the second estimation result on the assumption that the subject is not menopausal. Accordingly, by taking into account both cases, that is, menopausal or not menopausal, an estimation result with higher accuracy may be output. When the subject is a woman who is not menopausal at the current time point, the estimator 23 may simultaneously output the first estimation result on the assumption that the subject is menopausal and the second estimation result on the assumption that the subject is not menopausal.

### Supplementary Note

The information processing device 1 may not include all the functional blocks included in the controller 2 of the information processing device 1. For example, in the information processing system 100a, the terminal device 30 may have the function of the acquirer 21, and the information processing device 1 may receive the input information from the terminal device 30. Similarly, in each of the information processing system 100b, the information processing device 1 may receive input information from the terminal device 30.

For example, the information processing systems 100a and 100b may include the information processing device 1 in which the learning model 33 trained by another computer that is different from the information processing device 1 and has the function of the trainer 25 is installed. For example, in the information processing systems 100a and 100b, another computer other than the information processing device 1 or the terminal device 30 may have the function of the trainer 25. In the information processing systems 100a and 100b, another computer other than the information processing device 1 or the terminal device 30 may have the function of the estimator 23.

### Example of Implementation using Software

A control block of the information processing device 1 (the controller 2 in particular) may be implemented by a logic circuit (hardware) formed in an integrated circuit (IC chip) or the like, or may be implemented by software.

In the latter case, the information processing device 1 includes a computer that executes instructions of a program that is software implementing each function. The computer includes, for example, one or more processors, and also includes a computer-readable recording medium that stores the above program. In the computer, the processor reads the above program from the recording medium and executes the read program to achieve the object of the present disclosure. As the processor, a central processing unit (CPU) can be used, for example. As the recording medium, a “non-transitory tangible medium”, for example, a tape, a disk, a card, a semiconductor memory, a programmable logic circuit, or the like can be used in addition to a read only memory (ROM). The computer may further include a random access memory (RAM) for loading the above program. The above program may be supplied to the computer via any transmission medium (communication network, broadcast waves, and the like) that can transmit the program. One aspect of the present disclosure may be implemented in the form of data signals embedded in a carrier wave in which the above program is embodied by electronic transmission.

The invention according to the present disclosure has been described above based on various drawings and examples. However, the invention according to the present disclosure is not limited to each embodiment described above. That is, the invention according to the present disclosure can be modified in various ways within the scope described in the present disclosure, and embodiments obtained by combining technical means disclosed in the different embodiments as appropriate are also included in the technical scope of the invention according to the present disclosure. In other words, a person skilled in the art can easily make various variations or modifications based on the present disclosure. It is to be noted that these variations or modifications are included in the scope of the present disclosure.

### Conclusion

An information processing system according to a first aspect of the present disclosure includes: an acquirer configured to acquire medical information including first medical information including a medical image showing a bone of a subject and second medical information including feature information of the subject at a time point different from a time point at which the medical image is captured; and an estimator configured to estimate bone state information on a future or past bone state of the subject by inputting the medical information into a learning model trained using training data including a plurality of pieces of information each including a medical image showing a bone of a predetermined person and feature information of the predetermined person at a time point different from a time point at which the medical image is captured.

In an information processing system according to a second aspect of the present disclosure, in the first aspect above, at least one selected from the group consisting of a fracture, a bone density, a bone quality, a trabecular bone score, an occurrence probability of fracture, a possibility of osteoporosis, a drug efficacy, an incident occurrence, a trabecular number, a trabecular separation, a trabecular thickness, a trabecular orientation, and a connectivity density of bone of the subject may be included.

In an information processing system according to a third aspect of present disclosure, in the first or the second aspect above, the estimator may estimate the bone state information of the subject after a time shorter than a time interval between any two time points among the time point at which the first medical information is acquired and at least one time point at which the second medical information is acquired.

In an information processing system according to a fourth aspect of the present disclosure, in the first or the second aspect above, the estimator may estimate the bone state information after a time longer than a time interval between an earliest time point and a latest time point among the time point at which the first medical information is acquired and at least one time point at which the second medical information is acquired.

In an information processing system according to a fifth aspect of the present disclosure, in any one of the first to the fourth aspects above, the learning model may be a learning model trained using training data including pieces of medical information of the predetermined person acquired at a plurality of different time points.

In an information processing system according to a sixth aspect of the present disclosure, in any one of the first to the fifth aspects above, when the second medical information acquired by the acquirer includes pieces of medical information at two or more time points and data of any of the second medical information at the two or more time points and the first medical information is anomalous data, the estimator may input data excluding the anomalous data into the learning model.

In an information processing system according to a seventh aspect of the present disclosure, in any one of the first to the sixth aspects above, when the second medical information acquired by the acquirer includes pieces of medical information at two or more time points and data of any of the second medical information at the two or more time points and the first medical information is anomalous data, the estimator may replace the anomalous data with data obtained by approximating the anomalous data to other data to be input into the learning model.

In an information processing system according to an eighth aspect of the present disclosure, in any one of the first to the seventh aspects above, the estimator may input, into the learning model, data excluding data outside a range of a predetermined threshold from data included in the medical information acquired by the acquirer.

In an information processing system according to a ninth aspect of the present disclosure, in any one of the first to the eighth aspects above, when the subject is a woman who is not menopausal at a current time point, the estimator may output at least one selected from the group consisting of a first estimation result on an assumption that the subject is menopausal and a second estimation result on an assumption that the subject is not menopausal.

In an information processing system according to a 10th aspect of the present disclosure, in any one of the first to the ninth aspects above, the feature information may include at least one selected from the group consisting of a medical image, past bone state information, attribute information, a test numerical value, and a diagnostic result.

In an information processing system according to an 11th aspect of the present disclosure, in the 10th aspect above, the past bone state information may include at least one selected from the group consisting of a bone density and a trabecular bone score.

In an information processing system according to a 12th aspect of the present disclosure, in the 10th or the 11th aspect above, the attribute information may include at least one selected from the group consisting of an age, a sex, a height, a weight, a lifestyle habit, medication information, occupation information, blood test information, urine test information, saliva test information, a medical history, a family medical history of the subject, genetic information, menopause information, and a menopause estimation estimated based on hormone information.

In an information processing system according to a 13th aspect of the present disclosure, in any one of the 10th to the 12th aspects above, the estimator may input at least the attribute information as the medical information into the learning model.

In an information processing system according to a 14th aspect of the present disclosure, in any one of the 10th to the 13th aspects above, the test numerical value may include at least one selected from the group consisting of a bone density, Kellgren-Lawrence (KL) classification, a bone morphology angle, a muscle mass, mini mental state examination (MMSE), a blood test numerical value, a hepatic function marker, a uric acid level, and a malignant tumor marker.

In an information processing system according to a 15th aspect of the present disclosure, in any one of the first to the 14th aspects above, the first medical information may include an information element that is identical to the feature information included in the second medical information.

In an information processing system according to a 16th aspect of the present disclosure, in any one of the first to the 15th aspects above, the medical image may include at least one selected from the group consisting of an X-ray image, a computed tomography (CT) image, a magnetic resonance imaging (MRI) image, an ultrasonic image, and a positron emission tomography (PET) image.

In an information processing system according to a 17th aspect of the present disclosure, in any one of the first to the 16th aspects above, the estimator may estimate bone state information on states of a bone of the subject at a plurality of time points in future and in past.

In an information processing system according to an 18th aspect of the present disclosure, in the first aspect above, when the first medical information including a first medical image showing a trabecular bone of the subject is input into the learning model, the learning model may be trained using training data including a second medical image showing a trabecular bone of a predetermined person and trabecular bone information on a state of the trabecular bone of the predetermined person.

In an information processing system according to a 19th aspect of the present disclosure, in the first aspect above, when the first medical information including a first virtual image in which a color shade in a first medical image showing a trabecular bone of the subject is expressed for respective predetermined areas is input into the learning model, the learning model may be trained using training data including at least a second virtual image in which a color shade in a second medical image showing a trabecular bone of a predetermined person is expressed for respective predetermined areas.

In an information processing system according to a 20th aspect of the present disclosure, in the 18th aspect above, the trabecular bone information may be data related to a state of a trabecular bone of the predetermined person at a time point different from a time point at which the second medical image is captured, and the estimator may estimate future bone state information of the subject.

In an information processing system according to a 21st aspect of the present disclosure, in the 18th or the 19th aspect above, the trabecular bone information may be data related to a trabecular bone of the predetermined person at a time point at which the second medical image is captured, and the estimator may estimate current bone state information of the subject.

In an information processing system according to a 22nd aspect of the present disclosure, in any one of the 18th to the 20th aspects above, the medical information may include at least the first virtual image.

A terminal device according to a 23rd aspect of the present disclosure is configured to present, on a display, bone state information on a future or past state of a bone of a subject estimated by inputting, into a learning model trained using training data including a plurality of pieces of information each including a medical image showing a bone of a predetermined person and feature information of the predetermined person at a time point different from a time point at which the medical image is captured, medical information including first medical information including a medical image showing a bone of the subject and second medical information including feature information of the subject at a time point different from a time point at which the medical image is captured.

A method for controlling an information processing system according to a 24th aspect of the present disclosure includes: acquiring medical information including first medical information including a medical image showing a bone of a subject and second medical information including feature information of the subject at a time point different from a time point at which the medical image is captured; and estimating bone state information on a future bone state of the subject by inputting the medical information into a learning model trained using training data including a plurality of pieces of information each including a medical image showing a bone of a predetermined person and feature information of the predetermined person at a time point different from a time point at which the medical image is captured.

A method for controlling an information processing system according to a 25th aspect of the present disclosure is a control program for causing a computer to function as the information processing system according to any one of the first to the 22nd aspects above, and is a control program causing the computer to function as the acquirer and the estimator.

A recording medium according to a 26th aspect of the present disclosure is a computer-readable recording medium on which the control program according to the 25th aspect is recorded.

### REFERENCE SIGNS

1 Information processing device
21 Acquirer
23 Estimator
24 Outputter
32 Training data
33 Learning model
100a, 100b Information processing system
S11, S31 Acquiring step
S12, S32 Estimating step
S13, S33 Outputting step

1. An information processing system comprising:
an acquirer configured to acquire medical information comprising first medical information comprising a medical image showing a bone of a subject and second medical information comprising feature information of the subject at a time point different from a time point at which the medical image is captured; and
an estimator configured to estimate bone state information on a future or past bone state of the subject by inputting the medical information into a learning model trained using training data comprising a plurality of pieces of information each comprising a medical image showing a bone of a predetermined person and feature information of the predetermined person at a time point different from a time point at which the medical image is captured.

2. (canceled)

3. The information processing system according to claim 1, wherein the estimator estimates the bone state information of the subject after a time shorter than a time interval between any two time points among the time point at which the first medical information is acquired and at least one time point at which the second medical information is acquired.

4. The information processing system according to claim 1, wherein the estimator estimates the bone state information after a time longer than a time interval between an earliest time point and a latest time point among the time point at which the first medical information is acquired and at least one time point at which the second medical information is acquired.

5. The information processing system according to claim 1, wherein the learning model is a learning model trained using training data comprising pieces of medical information of the predetermined person acquired at a plurality of different time points.

6. The information processing system according of claim 1, wherein when the second medical information acquired by the acquirer comprises pieces of medical information at two or more time points and data of any of the second medical information at the two or more time points and the first medical information is anomalous data, the estimator inputs data excluding the anomalous data into the learning model.

7. The information processing system according of claim 1, wherein when the second medical information acquired by the acquirer comprises pieces of medical information at two or more time points and data of any of the second medical information at the two or more time points and the first medical information is anomalous data, the estimator replaces the anomalous data with data obtained by approximating the anomalous data to other data to be input into the learning model.

8. The information processing system according of claim 1, wherein the estimator inputs, into the learning model, data excluding data outside a range of a predetermined threshold from data comprised in the medical information acquired by the acquirer.

9. The information processing system according of claim 1, wherein when the subject is a woman who is not menopausal at a current time point, the estimator outputs at least one selected from the group consisting of a first estimation result on an assumption that the subject is menopausal and a second estimation result on an assumption that the subject is not menopausal.

10. The information processing system according of claim 1, wherein the feature information comprises at least one selected from the group consisting of a medical image, past bone state information, attribute information, a test numerical value, and a diagnostic result.

11. The information processing system according to claim 10, wherein the past bone state information comprises at least one selected from the group consisting of a bone density and a trabecular bone score.

12. (canceled)

13. The information processing system according of claim 10, wherein the estimator inputs at least the attribute information as the medical information into the learning model.

14. (canceled)

15. The information processing system according to claim 1, wherein the first medical information comprises an information element that is identical to the feature information comprised in the second medical information.

16. (canceled)

17. The information processing system according to claim 1, wherein the estimator estimates bone state information on states of a bone of the subject at a plurality of time points in future and in past.

18. The information processing system according to claim 1, wherein
when the first medical information comprising a first medical image showing a trabecular bone of the subject is input into the learning model, the learning model is trained using training data comprising a second medical image showing a trabecular bone of a predetermined person and trabecular bone information on a state of the trabecular bone of the predetermined person.

19. The information processing system according to claim 1, wherein
when the first medical information comprising a first virtual image in which a color shade in a first medical image showing a trabecular bone of the subject is expressed for respective predetermined areas is input into the learning model, the learning model is trained using training data comprising at least a second virtual image in which a color shade in a second medical image showing a trabecular bone of a predetermined person is expressed for respective predetermined areas.

20. The information processing system according to claim 18, wherein
the trabecular bone information is data related to a state of a trabecular bone of the predetermined person at a time point different from a time point at which the second medical image is captured, and
the estimator estimates future bone state information of the subject.

21. The information processing system according to claim 18, wherein
the trabecular bone information is data related to a trabecular bone of the predetermined person at a time point at which the second medical image is captured, and
the estimator estimates current bone state information of the subject.

22. The information processing system according to claim 19, wherein the medical information comprises at least the first virtual image.

23. (Canceled)

24. A method for controlling an information processing system, the method comprising:
acquiring medical information comprising first medical information comprising a medical image showing a bone of a subject and second medical information comprising feature information of the subject at a time point different from a time point at which the medical image is captured; and
estimating bone state information on a future or past bone state of the subject by inputting the medical information into a learning model trained using training data comprising a plurality of pieces of information each comprising a medical image showing a bone of a predetermined person and feature information of the predetermined person at a time point different from a time point at which the medical image is captured.

25. (canceled)

26. A non-transitory computer-readable recording medium recording a control program for causing a computer to function as the information processing system according to claim 1, the control program causing the computer to function as the acquirer and the estimator.
